SEQUENCE LISTING (2 sequences)

SEQ ID NO: 1 - DNA, 1236 bp, Trypanosoma congolense
atgccgcgcctgatgacacactccctgactgcgctgcttatgggctgcgcactgctcctgttcttaaattcatcccccgtgtttgcccaagagagcaatgcaacggaagaggatgtggaaagtagcagcgttccgactaagactgatgaggagaaggtttttattaggaatgtgacatgcgaggcggatatttatgcatccagagcgctgtgcgccttccccagactgatgaaagaggtggaacaggaacgacatgtggcgggggacaatatcaagggagatgtcgagaataaggcacaaaaggcacatgacgcagcagaggcgctacgaggcgccatttcgctcgccaaggacacacttaacgatccggagggaaagttggtgaaagtggcagacaacctggaagagatttccaaggaggatctggggctcgtgcagacgatggtggatgaagccaacaaggcgtctgaggaagtgggtgagctttacaaggaggccagcaagagaaccccgggagagggcaaatgttccggtgagagtggcataaagagcttttatggctgcggtgatctgaagggcatccgcttcagcaactacagtgacggctgcgggccactgggtatggacaacttattggggcacgtcggccccctggcgtgcaagaaaacgcctaattgtcatggcgctctgcgtaatggcttgctcctcacactttacgtgagggaatgggactacacgagacccctggtgaaagacgagtgtacggataaggatgagtggctcaaagacattgaggacgttggaagagccatgaaggatttgggtaataaaataaacgattttaacgctactctttcaaagatcgacaaatacattgatgttgtcgtgaaaattacagaaatggtaaaggatggaaagagttacgaggatatactgaaggctgttgaaggaatgaagagcgcaagcgacggatcatcgtcgatgctcaaaaccggtagtgaagaggatcccttcaacatctctgagcttatgaatgggagggagaccttctgggggaagtacagcatttggataatggcgggcgctggctccgctgtcggcctgctgctgctggtgacgcttctcttcgtgtgctgccgtcgccgggcttcgaggaagggcgacggcgatgccacgacaggggctgcgcctactgggaagggcacctacgccgatccgtacaaccctgacggctaa

SEQ ID NO: 2 - protein, 411 aa, Trypanosoma congolense
MPRLMTHSLTALLMGCALLLFLNSSPVFAQESNATEEDVESSSVPTKTDEEKVFIRNVTCEADIYASRALCAFPRLMKEVEQERHVAGDNIKGDVENKAQKAHDAAEALRGAISLAKDTLNDPEGKLVKVADNLEEISKEDLGLVQTMVDEANKASEEVGELYKEASKRTPGEGKCSGESGIKSFYGCGDLKGIRFSNYSDGCGPLGMDNLLGHVGPLACKKTPNCHGALRNGLLLTLYVREWDYTRPLVKDECTDKDEWLKDIEDVGRAMKDLGNKINDFNATLSKIDKYIDVVVKITEMVKDGKSYEDILKAVEGMKSASDGSSSMLKTGSEEDPFNISELMNGRETFWGKYSIWIMAGAGSAVGLLLLVTLLFVCCRRRASRKGDGDATTGAAPTGKGTYADPYNPDG

(19) United States
(12) Patent Application Publication
(10) Pub. No.: US 2013/0108660 A1
(43) Pub. Date: May 2, 2013

(54) ANTI-TRYPANOSOME THERAPEUTIC AND DIAGNOSTIC APPLICATIONS

(71) Applicants: Virginie Coustou Linares, Marcheprime (FR); Theo Baltz, Bordeaux (FR); Magali Thonnus, Bordeaux (FR)

(72) Inventors: Virginie Coustou Linares, Marcheprime (FR); Theo Baltz, Bordeaux (FR); Magali Thonnus, Bordeaux (FR)

(21) Appl. No.: 13/509,389

(22) PCT Filed: Nov. 10, 2010
(86) PCT No.: PCT/EP2010/067208

§ 371 Date: Nov. 29, 2012

(30) Foreign Application Priority Data
Nov. 13, 2009 (FR) 0958035

(51) Int. Cl. C07K 14/44; G01N 33/68; C07K 16/20; C12Q 1/02; A61K 39/005; A61K 45/06

(52) U.S. Cl. CPC C07K 14/44; A61K 39/005; A61K 45/06; C07K 16/20; C12Q 1/025; G01N 33/6893
USPC 424/191.1; 536/23.5; 530/350; 530/387.9; 530/359; 435/320.1; 435/7.1; 435/29; 435/7.94; 435/252.33; 435/348; 435/362; 435/254.2; 435/254.23; 435/254.21

(57) ABSTRACT

The present invention relates to the identification of nucleotide and peptide sequences of a novel HDL transporter located in the flagellar pocket of African trypanosome parasites, and to the use thereof for anti-trypanosomosis diagnostic, therapeutic and vaccine applications in man and in animals.

The present invention relates to the identification of nucleotide and peptide sequences of a novel protein of the flagellar pocket specific of African trypanosome parasites and to the use thereof for diagnostic and therapeutic applications and notably as a vaccine for immunizing human and/or nonhuman animals or as a therapeutic target in the fight against trypanosomosis or trypanosomiasis.

Trypanosomosis and trypanosomiasis are caused by several species of parasitic protozoa of the genus Trypanosoma, and African trypanosomes generally refer to trypanosomes belonging to the group Salivaria, which itself includes three principal sub-genera: Trypanozoon, Duttonella and Nannomonas.

Only the sub-genus Trypanozoon comprises, in addition to species infectious to animals, two species infectious to humans in whom they cause sleeping sickness. The other sub-genera include species that infect wild and domestic animals and are never infectious to humans, but which can have significant indirect health consequences. The sub-genus Trypanozoon consists of polymorphic trypanosomes (long and short or stumpy forms), with an optional free flagellum and a small kinetoplast in the subterminal (posterior) position. The species of this sub-genus are Trypanosoma (T.) brucei, T. evansi and T. equiperdum. T. brucei includes three subspecies: T. b. brucei, T. b. gambiense and T. b. rhodesiense, which are quite similar in morphological, antigenic and biochemical terms and which are distinguished by their infectious nature, their pathogenicity and their geographical distribution. T. brucei and its subspecies are transmitted by tsetse flies. T. evansi is transmitted to cattle, horses and camels by biting flies other than tsetse (Tabanidae) in Africa, South America and Southeast Asia. T. equiperdum has no invertebrate host (sexual transmission in horses). The latter two species extend far beyond areas with tsetse flies and are cosmopolitan. Their morphology is similar to that of T. brucei but they are monomorphic (long forms only).

Trypanosomes belonging to the sub-genus Duttonella are club shaped, with a round and broad posterior extremity and a body that narrows toward the anterior extremity. The kinetoplast is voluminous, round and in the terminal position; the undulating membrane is relatively undeveloped, narrow and terminates in a free flagellum. T. vivax and T. uniforme, species of parasites of wild and domestic ruminants, can be transmitted mechanically or by tsetse flies in which they colonize exclusively the proboscis and proventriculus.

Trypanosomes of the sub-genus Nannomonas are small (8-24 μm), and they have no free flagellum at any stage of their development. The average-size kinetoplast is in the subterminal or marginal position. The posterior extremity is round and the undulating membrane narrow. Their pathogenicity in Africa is significant for cattle, pigs and dogs. Their development in the tsetse fly takes place exclusively in the stomach and proboscis. The principal species are T. congolense and T. simiae. These trypanosomes are small with a round posterior extremity, a kinetoplast in the marginal position and a narrow undulating membrane. They do not have a free flagellum.

Domestic ruminants in Africa are primarily infected by three species of pathogenic trypanosomes, T. congolense, T. vivax and T. brucei, which are responsible for a pathology called nagana. Other animals are infected by another pathogenic trypanosome species, T. evansi, which is responsible for a pathology called surra. Trypanosomes are characterized by a large genetic diversity, which relates to their infectivity, virulence, pathogenicity, transmissibility and sensitivity to trypanocidal products.

T. congolense is the principal agent of bovine trypanosomosis in Africa, by its frequency and pathogenicity. It also adapts to various nonhuman animal species, and can thus indifferently parasitize bovids, suids, ovids, caprids, equids and canids.

T. brucei, and notably the subspecies Trypanosoma brucei gambiense, is probably the most widely known since it is responsible for the chronic form of sleeping sickness in man in Western and Central Africa. The subspecies Trypanosoma brucei brucei is a parasite of domestic and wild animals throughout Africa, but it is not infectious to man due to the lytic effect of apolipoprotein L, present in human serum, on the blood forms of these trypanosomes. The third subspecies is Trypanosoma brucei rhodesiense, which is the agent of sleeping sickness in its acute form in Africa.

Additionally, the subspecies T. evansi is transmitted to bovids, horses and camels and has significant economic repercussions in Africa, notably for the breeding of cattle and buffaloes.

Lastly, T. vivax is a parasite primarily of ungulates in tropical Africa and is transmitted by horseflies and gadflies.

Trypanosomes have a complex life cycle which includes various morphological forms. They have in general a fusiform body. They have a flagellum connected to the body by an undulating membrane. They reproduce asexually by binary fission. During an infection, the tsetse fly (Glossina sp.) injects into the dermis of the host at the puncture site the infectious metacyclics present in the mouthparts. The parasites multiply in the dermis at the inoculation point. A local reaction related to parasite multiplication in the dermis occurs, and the parasites give rise to blood forms. This stage can last from 1-3 weeks, for example, in the case of T. congolense. Then, the parasites invade the blood, the lymphatic system, in particular the lymph nodes, and various organs such as the liver, spleen, heart, kidneys and testicles, which then exhibit significant lesions. The tsetse becomes infected by and feeds on parasitized animals, and once infected it remains infectious throughout its life. In the case of T. brucei and T. congolense, the trypanosome undergoes in the insect a complex cycle involving dedifferentiation in the intestine into noninfectious procyclic forms. In the salivary glands or mouthparts, trypanosomes transform into adherent epimastigote forms which actively multiply. Their differentiation leads to the infectious stage represented by metacyclic forms, which divide no further.

The T. vivax cycle comprises no procyclic stage. It begins with flagellum attachment in the blood forms introduced by the tsetse. They differentiate into epimastigote forms, which proliferate and then differentiate into infectious metacyclics. The total duration of the cycle in the tsetse is roughly 5-10 days for T. vivax, 18 days for T. congolense and 30 days for T. brucei.

The sources of infection for domestic animals are other domestic animals or wild animals that are sick or are healthy carriers. The existence of the reservoir comes from the fact that certain species are relatively unreceptive to the infection, and relatively insensitive to the disease. Potential vectors vary by trypanosome species. T. congolense and T. brucei are transmitted exclusively by biological vectors such as tsetse flies, but T. vivax can also be transmitted by mechanical vectors such as biting flies (gadflies or stable flies). T. evansi is transmitted exclusively by mechanical vectors. Transmission efficiency depends on tsetse infection rates and host-vector interactions. Generally, trypanosomes that are infectious to animals have higher infection rates than trypanosomes that infect man, which contributes to the very wide distribution of animal trypanosomosis.

Analysis of trypanosomes by electron microscopy shows the existence of a roughly 15 nm coat covering the totality of the cell body of the parasite. This coat is present only on the surface of the blood and metacyclic forms. It is comprised essentially of a variable surface glycoprotein (VSG) with other membrane proteins in small quantities. VSGs form a very dense structure comprising a physical barrier between the plasma membrane and the host. The 3-D structure predicts that only a small part of the protein is exposed on the surface of the parasite. Thus, the principal role of the coat is to mask the invariant membrane antigens of the parasite by presenting several immunodominant motifs to the immune defenses of the host. The coat further protects blood forms against lysis by activation of the alternate complement pathway.

Currently, control of the disease rests primarily on the control of vectors by the use of insecticides whose environmental impact is far from being negligible. In terms of infection, control of trypanosomosis rests exclusively on the use of drugs, because these parasites escape the immune defenses of the host by expressing variable surface antigens. This phenomenon of antigenic variation thus has for now impeded all efforts to develop a novel antiparasitic vaccine. Only a few molecules with limited effect against the pathogenic agent, trypanosomes, called trypanocides, are available. These chemotherapy treatments have numerous limitations. Indeed, treatment of trypanosomosis involves several trypanocidal products discovered for the most part several decades ago. All these products have significant side effects and many resistant or multiresistant parasite strains have appeared.

In terms of diagnostics, techniques that are rapid, reliable, inexpensive and applicable in the field are lacking. Various techniques are currently used but they too have many limitations. Clinical diagnostics, which are generally limited to suspicion, rely on the observation of symptoms which are generally relatively unspecific. Indeed, infections with other parasites that can cause certain comparable symptoms are frequent in these regions. The detection, either direct or after enrichment, of trypanosomes in the blood, lymph and other fluids is carried out by microscopic examination of the preparation (fresh or stained with Giemsa), and while this technique makes it possible to confirm trypanosomosis it is relatively insensitive. Molecular techniques such as PCR are costly and require too great an investment notably in the context of epidemiological studies. Serological techniques are based on total lysates, are difficult to standardize and are sensitive but relatively unspecific. The search for specific antigens using ELISA have not succeeded as of yet.

In this context, the search for novel therapeutic targets enabling the development of an “anti-pathology” vaccine to reduce the harmful effects of parasitemia on the animal or novel trypanocide molecules is a priority.

### SUMMARY OF THE INVENTION

The Applicant has identified and characterized in T. congolense a protein designated FLP80 located at the level of the flagellar pocket and the early endocytic compartment of blood forms of the parasite. The principal field of application envisaged is the fight against trypanosomosis and the use of said protein for biotechnological purposes. Indeed, the novel FLP80 protein is useful for the vaccination of trypanosomosis caused by African trypanosomes.

According to the invention, the newly characterized peptide sequences are used for the production of diagnostic tests and for the preparation of vaccine and pharmaceutical compositions. Said protein and any antigenic polypeptide fragment of said protein are further useful for the production of antibodies specific of the parasites, for the purpose of diagnostics or passive immunization. Lastly, FLP80 constitutes a therapeutic target of choice for the development of trypanocidal agents or competitive inhibitors.

### BRIEF DESCRIPTION OF THE FIGURES

FIG. 1: represents the nucleotide sequence coding for the flagellar pocket protein FLP80;

FIG. 2: represents the peptide sequence corresponding to the flagellar pocket protein FLP80;

FIG. 3A: represents a western blot analysis of FLP80 expression during the parasitic cycle in procyclic forms (PCF), epimastigote forms (EMF) and blood forms (BSF) in relation to the expression of tubulin (TUB) during three parasitic stages;

FIG. 3B: represents a western blot analysis of FLP80 in blood forms (BSF) without and after treatment with an endoglycosidase (PNGase).

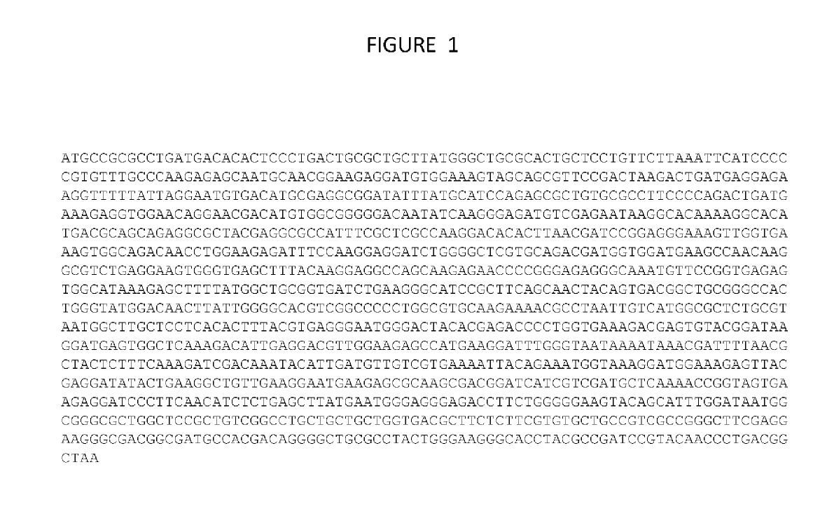

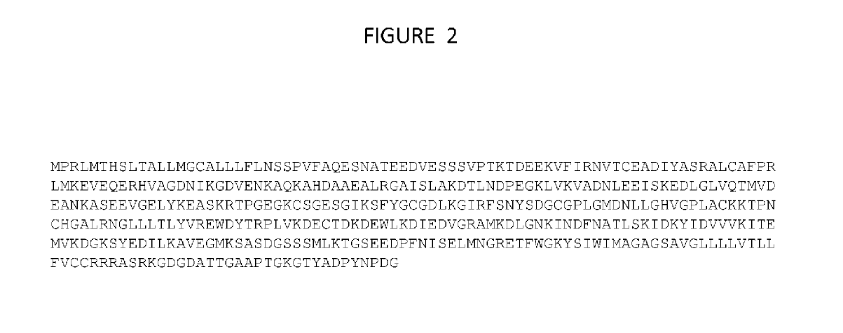

### DEFINITIONS

“African trypanosomes” refer to parasitic protozoa of the genus Trypanosoma belonging to the group Salivaria, which itself includes three principal sub-genera: Trypanozoon, Duttonella and Nannomonas, such as defined above. These have been described as African trypanosomes, but however are found today in Asia and South America as well as on the African continent. The most common African trypanosomes are Trypanosoma congolense, Trypanosoma vivax, Trypanosoma evansi and Trypanosoma brucei.

The terms “trypanosomosis” and “African animal trypanosomosis” (AAT) generally refer to infections of nonhuman animals caused by African trypanosomes, whereas the terms “trypanosomiasis” or “African trypanosomiasis” are used to refer to human infections also caused by African trypanosomes. For purposes of simplification, the terms trypanosomosis and trypanosomiasis are used indifferently herein.

### DETAILED DESCRIPTION OF THE INVENTION

An object of the present invention relates to a DNA or RNA molecule coding for a novel flagellar pocket protein, called FLP80, specific to African trypanosomes. Said novel DNA or RNA molecule comprises at least a nucleotide sequence as represented in the sequence SEQ ID NO: 1, a complementary sequence, an antisense sequence or a sequence equivalent to the sequence of SEQ ID NO: 1, and notably a sequence comprising an identity of at least 80%, 85%, 90%, 92%, 93%, 94% or 95% with the entire sequence SEQ ID NO: 1, or a nucleotide sequence able to hybridize with the sequence of SEQ ID NO: 1 under stringent hybridization conditions.

Stringent hybridization conditions refer to hybridization at a temperature of 65° C. overnight in a solution containing 0.1% SDS, 0.7% dried skimmed milk and 6×SSC, followed by washings at room temperature in 2×SSC—0.1% SDS and at 65° C. in 0.2×SSC—0.1% SDS.

The invention also relates to DNA or RNA fragments whose nucleotide sequence is identical, complementary, antisense or equivalent to the sequence of SEQ ID NO: 1, and notably DNA or RNA fragments having, for a sequence of roughly 30 to 100 contiguous nucleotides, or approximately 30 to 50, and preferably at least 30 contiguous nucleotides, at least 50%, at least 60%, at least 70%, or at least 80%, 85%, 90%, 92%, 93%, 94% or 95% homology with the sequence SEQ ID NO: 1, or a nucleotide sequence able to hybridize with the sequence SEQ ID NO: 1 under stringent hybridization conditions.

Nucleotide sequence refers to at least one strand of DNA or its complementary strand, or one strand of RNA or its antisense strand or the corresponding complementary DNA. The DNA sequence of SEQ ID NO: 1 corresponds to the messenger RNA sequence, given that that the thymine (T) in DNA is replaced by uracil (U) in RNA.

According to the invention, two nucleotide sequences are said to be equivalent with respect to each other as a result of natural variability, notably spontaneous mutation of the species from which they were identified, or induced variability, as well as homologous sequences, with homology being defined below. Variability refers to any spontaneous or induced modification of a sequence, notably by substitution and/or insertion and/or deletion of nucleotides and/or nucleotide fragments, and/or extension and/or shortening of the sequence at at least one end, or unnatural variability that may result from the genetic engineering techniques used. This variability can be expressed by modifications of any starting sequence, regarded as a reference, and can be expressed by a degree of homology in relation to said reference sequence.

Homology characterizes the degree of identity of two compared nucleotide (or peptide) fragments; it is measured by percent identity, which is notably determined by direct comparison of nucleotide (or peptide) sequences in relation to reference nucleotide (or peptide) sequences. The invention has as an object a Trypanosoma congolense protein, called FLP80, with an apparent molecular weight of roughly 40 kDa, as well as the antigenic peptide fragments or an immunological equivalent of said protein. It was identified in a membrane protein extract from blood forms of the parasite by mass spectrometry analysis. The sequence of flagellar protein FLP80 comprises 411 amino acids, and is represented in the sequence SEQ ID NO: 2. Analysis of the sequence shows the presence of a signal peptide and a transmembrane domain in positions 354 to 379. The sequence is cysteine-rich but no particular motif is present. Furthermore, the Applicant has shown that FLP80 is expressed differentially during the trypanosome cycle. Indeed, as shown in the experimental section, in Example 2 below, FLP80 is not detected in procyclic forms (PCF), but it is detected in epimastigote forms (EMF) with a molecular weight of approximately 60 kDa and in blood forms (BSF) with a molecular weight of 65 kDa. Treatment with endoglycosidases show that the size difference between the molecular weight observed in a western blot and the theoretical molecular weight is due to heavy glycosylation of the protein. Moreover, FLP80 is particularly abundant in blood forms. More precisely, FLP80 is present in 2.7·10⁵ specimens per cell. Its immunolocalization in blood forms shows that the protein is found in the flagellar pocket and the endocytic compartment (Example 3).

This protein is expressed in blood forms of the parasite, and is thus present throughout the infection, which represents an advantage in relation to vaccine candidates or diagnostics developed to date which have been demonstrated only in parasitic forms found in the insect. Indeed, it is not certain that such candidates are expressed during the infection, whereas the presence of the candidate in the blood of the infected mammalian host is a precondition necessary to its use notably for the establishment of a diagnostic test.

Immunological equivalent refers to any polypeptide or peptide able to be recognized immunologically by antibodies directed against FLP80.

The invention further relates to any fragment of FLP80, and more particularly any antigenic peptide fragment specifically recognized by anti-African trypanosome antisera.

Said protein and protein fragments of the invention can comprise modifications, notably chemical modifications that do not alter their immunogenicity. The peptides can be obtained by chemical synthesis, lysis of FLP80 or by genetic recombination techniques.

According to a second aspect, the present invention relates to a novel anti-parasite strategy, wherein FLP80 is used as a therapeutic target. More precisely, this novel anti-parasite strategy consists in using the novel flagellar protein FLP80 as a therapeutic target for the internalization of a trypanocidal agent or to inhibit the growth or multiplication of infectious parasites. Since FLP80 is highly abundant in the flagellar pocket membrane, it indeed constitutes a therapeutic target of choice for the internalization of trypanocidal molecules in parasites. The flagellar pocket is the only site of exchange between the parasite and the external environment and is not, in contrast with the rest of the cell body, covered with a dense and impenetrable coat of variable surface glycoproteins (VSG). Very few flagellar proteins have been described to date in trypanosomatids and the only known examples are present in low numbers per cell. This membrane protein, considering its abundance, likely covers a large portion of the flagellar pocket and appears essential to the parasite's survival in the infected mammalian host. Consequently, in relation to the therapeutic targets considered to date, FLP80 has numerous advantages in terms of abundance, localization, function and specificity, which make it possible to envisage a strategy aimed at blocking this protein's function. According to this aspect of the present invention, it has been notably discovered that the FLP80 membrane protein corresponds to an active transporter of high-density lipoproteins (HDL). More precisely, the FLP80 substrate was identified by affinity chromatography with mouse serum proteins as the HDL fraction (identification of apolipoproteins A1 and E by mass spectrometry).

Consequently, a further object of the present invention concerns apolipoproteins A1 or E, and notably a therapeutic complex comprising an apolipoprotein A1 or E complexed in a functional manner with a toxic molecule and able to bind to the FLP80 transporter. Coupling the FLP80 apolipoprotein ligand with a toxic therapeutic molecule makes it possible to internalize, and thus to strongly concentrate, such toxic molecules in the parasite via the special interface of the flagellar pocket.

Moreover, the inventive use of FLP80 as a transporter of toxic molecules represents an advantage in relation to a traditional long-term vaccine strategy. Since the concentration of toxic molecules in and around the infectious agent concerned is always a major problem in any given treatment, the presence of FLP80 in large numbers at the special site of exchange comprised by the flagellar pocket makes it possible to very heavily concentrate a toxic molecule and thus to increase the treatment's chances of success, to limit the risks of toxicity for the host and to reduce the treatment's costs.

Among the compounds with trypanocidal activity and able to be coupled with the apolipoprotein ligand and internalized in the parasite via FLP80, mention may be made of trypanocidal agents such as diamidine (pentamidine or pentamidine mesylate, diminazene or diminazene aceturate), arsenic derivatives such as Melarsoprol®, melarsomine, eflornithine (DMFO), arsobal, MelBdm, nitrofuran derivatives such as nifurtimox (5-nitrofuran), ornithine analogs (Eflornithine® or difluoromethylornithine), phenanthridine (isometamidium or Homidium®), a polysulfonated naphtha-urea such as Suramin®, an anti-malignancy agent such as quinapyramine, buthionine sulfoximine (BSO), azaserine, 6-diazo-5-oxo-norleucine (DON) and/or acivicin.

The toxic molecules are coupled in a functional manner with the apolipoprotein ligand in such a way as to enable (1) the binding of the FLP80 complex with an affinity of the same order as the uncoupled apolipoprotein ligand, and (2) the internalization and concentration of the trypanocide complex in the trypanosome parasite.

As a functional equivalent of the apolipoprotein-toxic molecule complex, any molecule having a binding affinity for the membrane protein FLP80 similar to the apolipoprotein substrate can be used. Mention may be made, for example, of an antibody such as described below directed specifically against FLP80 linked to a toxic trypanocide molecule able to cause, after binding with FLP80, the internalization of the toxic molecule in the trypanosome.

Consequently, according to the present invention, the apolipoprotein-toxic molecule complex or a functional equivalent is used as a veterinary composition against trypanosomosis. Such trypanocidal veterinary compositions comprise the apolipoprotein substrate coupled in a functional manner with a trypanocidal agent such as described above in a therapeutically sufficient quantity to treat and/or prevent African trypanosomosis in animals or humans. Also, the trypanosomosis treatment methods comprise the obtention of an apolipoprotein complex substrate or equivalent, the coupling of the substrate to a toxic agent or trypanocide, and the administration of the trypanocide complex thus obtained to a human and/or a nonhuman animal having been or likely to be infected by African trypanosomes.

Methods for determining the binding of the apolipoprotein substrate or equivalent, and/or a trypanocide complex of the invention on membrane proteins such as FLP80 are well-known to the person skilled in the art and can be achieved, for example, by competitive inhibition experiments between the apolipoprotein A1 or E ligand and the trypanocide complex or by the Biacore™ technology for studying real-time binding without having to label the substrate or FLP80. This technique uses a biosensor of surface plasmon resonance which detects mass variations on the surface of a sensor chip on which the apolipoprotein ligand or the complex is immobilized covalently or non-covalently, and wherein FLP80 is injected by a microfluidic system in a continuous stream of buffer on the surface of the sensor chip. Real-time analysis makes it possible to determine the kinetics of the association and dissociation constants and to deduce therefrom the value of the affinity constant.

Also, internalization of the trypanocide complexes can be measured on cell lines expressing recombinant FLP80 and treated with the trypanocide complex comprising a radiolabeled apolipoprotein substrate coupled in a functional manner with a toxic agent, and the radioactivity associated with the cells can be measured.

Alternatively, another object of the present invention relates to competitive inhibitors of the binding of the natural substrate to FLP80 protein. Such competitive inhibitors are, for example, a truncated apolipoprotein A1 or E, i.e., comprising 10-80% of the size of natural apolipoproteins A1 and E. Specific antibodies of the FLP80 binding site can also constitute competitive inhibitors of the binding of natural substrate on FLP80, and thus inhibit the growth of and destroy the infectious trypanosomes.

The present invention further relates to a method for screening trypanocide complexes able to be internalized via FLP80 in infectious trypanosomes and thus able to destroy infectious trypanosomes. Alternatively, the present invention concerns a method for screening FLP80 inhibitors able to inhibit the infectious cycle of trypanosomes, their growth or their multiplication in the infected nonhuman animal host. The methods of the invention comprise a step of evaluation of the capacity of said molecules or agents to be internalized and to concentrate in the parasites in order to induce their destruction and/or to inhibit the activity or the function of FLP80 and/or to stop the infectious cycles of the parasites and/or to induce a reduction of their multiplication or their growth.

The screening method of the invention thus comprises an African trypanosome growth/multiplication inhibition test. The trypanocidal agents screened by the method such as defined above are characterized by their capacity to inhibit or modulate the growth or multiplication of infectious African trypanosomes.

Consequently, compositions comprising trypanocidal agents or FLP80 inhibitors as therapies against trypanosomosis, or for the preparation of a pharmaceutical composition to treat and/or prevent disease, as well as methods for treating and/or preventing trypanosomosis comprising the administration of such trypanocidal agents, inhibitor molecules or compositions also form an integral part of the invention.

According to a third aspect, a further object of the present invention resides in a functional expression cassette, notably in a cell from a prokaryotic or eukaryotic organism, enabling the expression of DNA coding for the totality or a fragment of FLP80 protein. In particular, a DNA fragment such as defined above is placed under the control of the elements required for its expression. Said protein or protein fragments thus expressed are recognized by anti-African trypanosome antisera.

Generally, any cell from a prokaryotic or eukaryotic organism can be used in the context of the present invention. Such cells are known to the person skilled in the art. As examples, mention may be made of cells from a eukaryotic organism, such as mammalian cells, notably Chinese hamster ovary (CHO) cells, insect cells or fungal cells, notably unicellular or yeast cells, notably from Pichia, Saccharomyces, Schizosaccharomyces and particularly selected from the group comprised of Saccharomyces cerevisiae, Schizosaccharomyces pombe, Schizosaccharomyces malidevorans, Schizosaccharomyces sloofiae and Schizosaccharomyces octosporus. Similarly, among cells from prokaryotic organisms, mention may be made, without constituting a limitation in any way, of cells of a strain of Escherichia coli (E. coli) or enterobacteria cells. The cell can be wild-type or mutant. Mutations are described in the literature available to the person skilled in the art. Preferably, an E. coli cell is used, such as BL21 (DE3), for example.

The expression cassette of the invention is intended for the production, for example in E. coli, of FLP80 or fragments of said protein recognized by anti-African trypanosome antisera. Such antisera come from animals having contracted a recent or old infection by T. congolense, and which have immunoglobulins that specifically recognize FLP80. Also, FLP80 can be recognized by other antibodies such as, for example, monoclonal or polyclonal antibodies obtained by immunization of varied species with the aforesaid natural protein, the recombinant protein, or the fragments or peptides thereof.

Flagellar protein FLP80 refers to the membrane antigen of T. congolense, produced notably by the genetic recombination techniques described in the present application, or any fragment or mutant of said antigen on the condition that it is immunologically reactive with antibodies directed against the FLP80 protein of said parasites.

Advantageously, said proteins have an amino acid sequence with a degree of homology of at least 70%, at least 80%, preferably at least 85% or 90%, and most preferably at least 92%, 93%, 94% or at least 95% in relation to the sequence SEQ ID NO: 2. In practice, one such equivalent can be obtained by deletion, substitution and/or addition of one or more amino acids of the native or recombinant protein. It is within the means of the person skilled in the art to carry out these modifications by known techniques without affecting immunological recognition.

In the context of the present invention, FLP80 can be modified in vitro, notably by deletion or addition of chemical groups such as phosphates, sugars or myristic acids, so as to improve its stability or the presentation of one or several epitopes.

The expression cassette of the invention enables the production of FLP80, having the amino acid sequence as specified above, and fragments of said protein, which can advantageously be fused with an exogenous element able to contribute to its stability, purification, production or recognition. The choice of one such exogenous element is within the means of the person skilled in the art. It can be notably a hapten or an exogenous peptide.

The expression cassette of the invention comprises the elements required for the expression of said DNA fragment in the cell under study. “Elements required for the expression” refer to all of the elements that enable the transcription of the DNA fragment into messenger RNA (mRNA), such as transcription promoter sequences (CMV promoter, for example) and terminator sequences, as well as elements enabling the translation of mRNA into protein.

The present invention further extends to vectors comprising an expression cassette of the invention. It can also be a plasmid vector capable of autonomous replication and in particular multiplication. It can be a viral vector and notably a baculovirus-derived vector, more particularly intended for expression in insect cells, or an adenovirus-derived vector for expression in mammalian cells.

The present invention also relates to a cell from a prokaryotic or eukaryotic organism, comprising an expression cassette, either integrated in the cell genome or inserted in a vector.

The present invention also concerns a method for preparing FLP80, or fragments of said protein, wherein: (i) a cell from a prokaryotic or eukaryotic organism, comprising the expression cassette of the invention, is cultivated under suitable conditions; and (ii) the protein expressed by said organism is recovered.

According to a fourth aspect, the invention relates to monoclonal or polyclonal antibodies obtained by immunological reaction of a nonhuman animal organism with an immunogenic agent comprised of natural or recombinant FLP80, and peptide fragments thereof, such as defined above. Antibody production techniques are well-known to the person skilled in the art. As examples, the polyclonal antibodies of the present invention can be generated by injecting FLP80 or an antigenic peptide fragment thereof into rabbits or mice in order to immunize them. The rabbit or mouse polyclonal sera thus obtained are tested for their reactivity by indirect ELISA.

According to a fifth aspect, the present invention has as an object an active immunotherapeutic composition, notably a vaccine preparation, comprising natural or recombinant FLP80, one or more antigenic peptide fragments thereof, such as defined above, and optionally a suitable excipient and/or adjuvant.

The vaccine or veterinary compositions of the invention are intended to treat and/or prevent an infection by African trypanosomes in a human and/or a nonhuman animal.

More particularly, the compositions of the inventions are intended to treat and/or prevent infection by T. congolense. African trypanosomosis, 75% of which are due to T. congolense, causes in animals syndromes of variable gravity, ranging from acute infection with mortality in 3 to 4 weeks to chronic infection lasting months or even years. The chronic progression, characterized by intermittent parasitemias, is the most frequent in African cattle. The disease begins with a hyperthermia phase, and then two to three weeks after the infecting bite the number of red blood cells and hemoglobin and hematocrit levels drop, reflecting anemia, which is the major symptom of trypanosomosis. Chronically infected animals consume less feed, become cachectic, their growth slows, and negative effects on reproduction are observed. Trypanosomosis anemia is established in two phases. During the initial phase, anemia is accompanied by parasitemia and results primarily from extra-vascular hemolysis: red blood cells are destroyed by the phagocyte system in the spleen, liver, circulating blood and bone marrow. Eventually, anemia results in bone marrow dysfunction.

Said veterinary vaccine compositions can be provided in the form of an antigenic vaccine and thus comprise a therapeutically effective amount of natural or recombinant FLP80, or the antigenic peptide fragments thereof, such as described above.

The vaccine compositions can be provided in the form of DNA vaccines and can thus comprise an expression cassette, a vector, a cell from a prokaryotic or eukaryotic organism such as defined above, able to express FLP80, or the antigenic peptide fragments thereof, and/or a combination thereof.

The vaccines of the present invention can be monovalent vaccines comprising an effective amount of natural or recombinant FLP80, and/or the antigenic peptide fragments thereof and/or the nucleotide sequences coding for said peptide peptides or fragments.

Said monovalent vaccine prevents the infestation and thus the expression of the disease.

If said vaccine does not prevent the infestation but only the expression of the disease, it could be called an “anti-disease” vaccine. In this case, and given that differential diagnosis with other blood parasitoses is currently not systematic, the use of multivalent vaccines combining the so-called “anti-disease” vaccine with antigens of other trypanosomes and/or other therapeutic active agents and/or other vaccines commonly used in disease prevention is particularly advantageous according to the present invention.

Thus, the vaccines of the present invention can be monovalent vaccines combining one or more natural or recombinant proteins and/or peptide fragments and/or nucleotide sequence coding for said peptides and peptide fragments of one or more trypanosome species, and preferably derived from one or more similar or different trypanosome species.

Said trypanosome-derived antigenic peptides, fragments or antigenic peptide cocktails are, for example, sialidases, trans-sialidases, tubulins, proteases, lipases or also other flagellar proteins.

As examples of trans-sialidases able to be incorporated into multivalent vaccines, mention may be made of the trans-sialidases of T. cruzi, T. congolense, T. vivax, T. evansi, T. brucei, T. rhodesiense and/or T. gambiense. Certain trans-sialidases of T. congolense, among others, are described in international application WO2004/55176 or by Tiralongo E. et al. (JBC vol. 278, No. 26, pp 23301-10, 2003). More precisely, mention may be made of T. cruzi trans-sialidase chains A and B as deposited in GenBank under numbers GI:29726491, GI:29726490, GI:29726489 and GI:29726488. It is also advantageous to use inactive mutated forms of trans-sialidases. In this respect, mention may be made of the mutant T. cruzi trans-sialidases described in international application WO2007/107488, for example, which conserve less than 20% of their sialidase and transferase enzymatic activity.

As examples of trypanosome-derived tubulins, mention may be made of T. brucei alpha-tubulin (deposited in GenBank under accession number AAA30262.1), T. brucei beta-tubulin (deposited in GenBank under accession number AAA30261.1), T. brucei epsilon-tubulin (deposited in GenBank under accession number EAN77544.1), T. brucei TREU927 epsilon-tubulin (referenced in NCBI under numbers XP_{—}822372.1 and XP_{—}829157.1), T. brucei delta-tubulin (deposited in GenBank under accession number EAN80045.1), T. brucei zeta-tubulin (referenced in NCBI under number XP_{—}001218818.1) or the T. brucei tubulins described in international application WO2008/134643.

As examples of trypanosome-derived flagellar proteins, mention may be made of the T. brucei flagellar protein described in international application WO2002/19960 or the T. congolense flagellar protein described in the Applicant's French application filed on 13 Nov. 2009 under number FR09/58035. Further mention may be made of the T. brucei TREU927 flagellar protein or flagellar-like proteins (referenced in NCBI under numbers XP 847376.1; XP_{—}847374.1; XP_{—}847295.1; XP_{—}843961.1; XP_{—}847377.1), the T. brucei flagellar protein TB-44A (deposited in GenBank under accession number AAZ13310.1), the T. brucei flagellar protein TB-24 (deposited in GenBank under accession number AAZ13308.1) and the T. brucei flagellar protein deposited in GenBank under accession number AAZ13311.1.

As examples of proteases, mention may be made of trypanosome cysteine proteases such as T. congolense congopain or trypanopain-Tc, T. rhodesiense rhodesain and T. cruzi chagasin or cruzipain.

The vaccines of the present invention, whether monovalent or multivalent, can further comprise adjuvants in order to increase antigenic response. Adjuvants are well-known to the person skilled in the art. As examples of adjuvants, mention may be made of vitamin E, aluminum gels or salts such as aluminum hydroxide or aluminum phosphates, metal salts, saponins, polyacrylic acid polymers such as Carbopol®, nonionic block polymers, fatty acid amines such as pyridine and DDA, dextran-based polymers such as dextran sulfate and DEAE-dextran, liposomes, bacterial immunogens such as LPS, peptidoglycans or MDP.

The nonhuman animals able to be treated include, for example, bovids, ovids, felids, suids, camelids and/or canids.

Alternatively, the vaccines can comprise an effective therapeutic quantity of a monoclonal or polyclonal antibody as described below.

The multivalent vaccines of the present invention can further contain antigens of other blood parasitoses derived, for example, from protozoa such as Theileria parva, T. annulate, Babesia bigemina and B. divergens to treat and/or prevent trypanosomes and theileriosis, anaplasmosis and/or babesiosis.

These can be further combined with other standard vaccines used for the prophylaxis and/or treatment of parasitoses in the target areas, namely against foot-and-mouth disease, clostridiosis, plague, catarrhal fever, contagious bovine pleuropneumonia (CBPP), blackleg, pasteurellosis and/or sheep pox.

The vaccines of the present invention are particularly useful for treating and/or preventing trypanosomosis-induced pathogeneses such as anemia, degradations in general health, weight loss and/or immunosuppression in man or nonhuman animals.

The monovalent or multivalent vaccines can also be administered in combination with antiparasitic agents, anti-infective agents and/or symptomatic agents.

Antiparasitic agents include, for example, trypanocidal drugs such as diamidines (pentamidine or pentamidine mesylate, diminazene or diminazene aceturate), arsenic derivatives such as Melarsoprol®, melarsomine, eflornithine (DMFO), arsobal, MelBdm, nitrofuran derivatives such as nifurtimox (5-nitrofuran), ornithine analogs (Eflornithine® or difluoromethylornithine), phenanthridine (isometamidium or Homidium®), a polysulfonated naphtha-urea such as Suramin®, an anti-malignancy agent such as quinapyramine, buthionine sulfoximine (BSO), azaserine, 6-diazo-5-oxo-norleucine (DON) and/or acivicin. When the vaccines are administered in combination with antiparasitic agents, the latter are preferably administered before and/or simultaneously and/or after the monovalent or multivalent vaccines described above. Other nonspecific antiparasitic agents for trypanosomes are well-known in the field, and are administered before and/or simultaneously and/or after the vaccines of the invention. Among these, mention may be made of avermectins (ivermectin, abamectin, doramectin, eprinomectin and selamectin), pyrethrins (deltamethrin, etc.) and/or anthelminthic antiparasitic agents (oxibendazole, piperazine, flubendazole).

As examples of anti-infective agents, mention may be made of antibiotics such as β-lactams, fosfomycin, glycopeptides or polypeptides with antibiotic activity, bacitracin, aminoglycosides, macrolides, lincosamides, streptogramins, tetracyclines, phenicols, fusidic acid or quinolones.

Symptomatic agents are, for example, anti-anemia agents such as iron, vitamin B12, folic acid or calcium levofolinate; or hepatoprotective agents such as flavonoid complexes (silymarin, silibinin, etc.), curcuma, Desmodium adscendens and/or Chrysanthellum americanum (carbon).

Non-steroidal anti-inflammatory drugs (NSAIDs) can include, among others, oxicams (meloxicam, piroxicam and/or tenoxicam), salicylate derivatives (methyl salicylate and acetylated lysine), 2-arylpropionic acids (profens), indole sulfonamide derivatives, selective COX-2 NSAIDs (celecoxib, etoricoxib, etc.), phenylbutazone, niflumic acid and/or fenamic acids.

According to a sixth aspect, the present invention relates to probes or primers specific of African trypanosome, and the use thereof in diagnostic tests.

The term “probe” as used in the present invention refers to DNA or RNA comprising at least one nucleotide sequence enabling hybridization with nucleic acids with at least one nucleotide sequence such as represented in the sequence SEQ ID NO: 1, a complementary sequence, an antisense sequence or an equivalent sequence to said sequence, and notably a sequence with five to 100 contiguous nucleotides that is at least 50%, preferably at least 60%, at least 70% or at least 85% homologous to the sequence SEQ ID NO: 1, or to a synthetic oligonucleotide enabling such hybridization, unmodified or comprising one or more modified bases such as inosine, methyl-5-deoxycytidine, deoxyuridine, dimethylamino-5-deoxyuridine, diamino-2,6-purine, bromo-5-deoxyuridine or any other modified base. Similarly, these probes can be modified at the sugar, namely the replacement of at least one deoxyribose with a polyamide, or at the phosphate group, for example its replacement by esters notably selected from diphosphate, dialkyl and arylphosphonate esters and phosphorothioate esters.

The probes can be much shorter than the sequence identified in the sequence SEQ ID NO: 1. In practice, such probes comprise at least five nucleotides, advantageously between five and 50 nucleotides, preferably roughly 20 nucleotides, having a hybridization specificity under conditions established to form a hybridization complex with the DNA or RNA having a nucleotide sequence as previously defined. The probes of the invention can be used for diagnostic purposes, as capture and/or detection probes.

The primers of the invention comprise a sequence of five to 30 consecutive nucleotides of the sequence SEQ ID NO: 1, and have a hybridization specificity under predetermined conditions to initiate enzymatic polymerization, for example in an amplification technique such as the polymerase chain reaction (PCR), in an extension process such as sequencing, in a reverse transcription method or the like.

According to a seventh aspect, the invention comprises detection and/or monitoring reagent as well as to a method and kits for diagnosing infections by T. congolense. The trypanosome detection reagents or diagnostic kits comprise as the reactive substance at least one monoclonal or polyclonal antibody as described above. Alternatively, the trypanosome detection reagents or diagnostic kit comprise a probe and/or primer to detect and/or identify African trypanosomes in a biological sample, notably a capture probe and/or a detection probe, with one and/or the other as defined above.

According to a preferred embodiment, the present invention relates more particularly to a method of immunodetection and immunolocalization of FLP80 during the parasitic cycle.

The reagent used for immunodetection and immunolocalization can be bound directly or indirectly to a suitable solid support. The solid support can be notably in the form of a cone, tube, well, bead or the like.

The term “solid support” as used herein includes all the materials on which a reagent can be immobilized for use in diagnostic tests. Natural or synthetic materials, chemically modified or not, can be used as solid supports, notably polysaccharides such as cellulose-based materials, for example paper, cellulose derivatives such as nitrocellulose and acetate; polymers such as vinyl chloride, polyethylene, polystyrene, polyacrylate or copolymers such as vinyl chloride and propylene polymers, vinyl chloride and vinyl acetate polymers, styrene-based copolymers, natural fibers such as cotton and synthetic fibers such as nylon.

The reagent can be bound to the solid support directly or indirectly. Directly, two approaches are possible, either by adsorption of the reagent on the solid support, i.e., by noncovalent bonds (mainly hydrogen, van der Waals or ionic bonds) or by establishment of covalent bonds between the reagent and the support. Indirectly, an “anti-reagent” compound able to interact with the reagent in order to immobilize the unit on the solid support can be bound beforehand (by adsorption or covalence) to the solid support. As an example, mention may be made of an anti-FLP80 antibody, on the condition that it is immunologically reactive with a different part of the protein than that participating in the sera antibody recognition reaction; a ligand-receptor system, for example, by grafting on the FLP80 protein a molecule such as a vitamin, and by immobilizing the corresponding receptor on the solid phase (for example the biotin-streptavidin system). Indirect approaches also include the preliminary grafting or fusion by genetic recombination of a protein, or a fragment of said protein, or a polypeptide, at one end of the FLP80 protein, and immobilization of the latter on the solid support by passive adsorption or covalence of the grafted or fused protein or polypeptide.

Capture probes can be immobilized on a solid support by any suitable means, i.e., directly or indirectly, for example by covalence or passive adsorption. Detection probes are labeled by means of a label selected from radioactive isotopes, enzymes notably selected from peroxidase and alkaline phosphatase, and those able to hydrolyze a chromogenic, fluorogenic or luminescent substrate, chemical chromophores, chromogenic, fluorogenic or luminescent compounds, nucleotide basic analogs, and biotin.

The probes of the present invention used for diagnostic purposes can be implemented in any known hybridization techniques, and notably so-called “dot-blot” techniques; Southern blot; northern blot, which is a technique identical to the Southern blot technique but which uses RNA as the target; and the sandwich technique.

The method of immunodetection and/or immunolocalization of an African trypanosome infection in a biological sample, such as a blood sample from a nonhuman animal, consists in bringing together said sample with a reagent such as defined above, under conditions enabling a possible immunological reaction, then the detection of the presence of an immune complex with said reagent. Preferably, said method implements an immune serum comprising polyclonal antibodies obtained, for example, from mice immunized by the recombinant protein expressed in E. coli.

According to a preferential embodiment, the present invention has as an object a method for diagnosing trypanosomosis comprising the detection of FLP80 as a circulating antigen during the infection, and the detection of the presence of trypanosome parasites in cattle found in an endemic region in order to apply the suitable treatment.

As a nonrestrictive example, mention may be made of the one- or multi-step ELISA detection technique, which consists in reacting a first specific monoclonal or polyclonal antibody for the antigen sought, bound to a solid support, with the sample, and revealing the possible presence of an immune complex thus formed by a second antibody labeled by any suitable label known to the person skilled in the art, notably a radioactive isotope, an enzyme, for example peroxidase or alkaline phosphatase or the like, by so-called competition techniques well-known to the person skilled in the art.

Finally, according to this aspect, the present invention has as an object a kit for veterinary use for diagnosing trypanosomosis in a biological sample, comprising a probe or a primer, or an antibody such as described above as well as a reagent for detecting an immunological reaction. The kits of the present invention comprise at least one compartment for an optionally sterile packaging comprising an effective therapeutic quantity of a reagent such as described above, as well as instructions relating to the protocol for implementing the veterinary diagnostics of the invention.

### EXAMPLES

### Example 1

### Demonstration of FLP80 Expression During the Parasitic Cycle

5·10⁶ cells of the T. congolense strain IL3000 for procyclic forms (PCF), epimastigote forms (EMF) and blood forms (BSF) were subjected to western blot analysis with an antiserum directed against FLP80 or tubulin (load control).

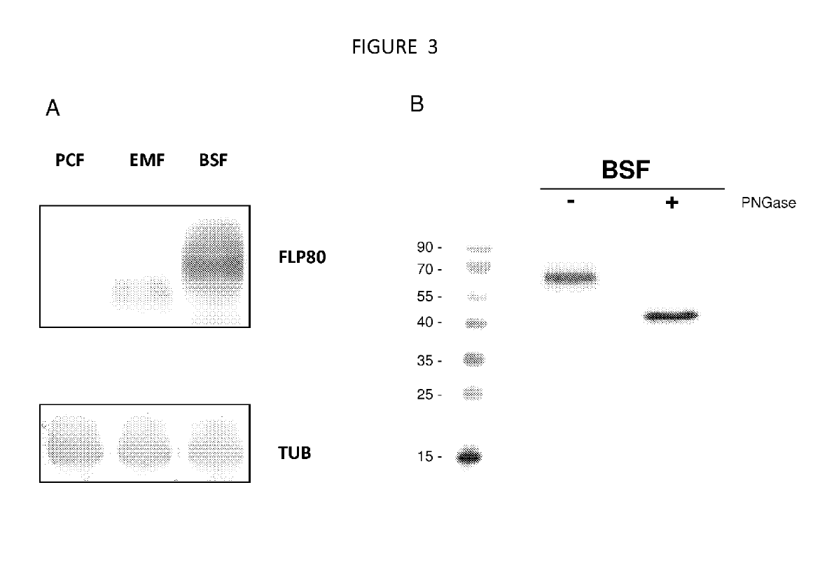

As shown on the western blot in FIG. 3, no signal was detected in procyclic forms, a signal corresponding to a molecular weight of 60 kDa was detected in epimastigote forms and a still stronger signal was detected corresponding to a molecular weight of 65 kDa. A constant signal was detected in the three parasitic stages for tubulin.

### Example 2

### Immunolocalization of FLP80 in T. congolense Blood Forms

T. congolense blood forms were labeled with DAPI which stains nuclear and mitochondrial (kinetoplast) DNA, an FLP80 antiserum or tomato lectin which stains the flagellar pocket and the early endosome. The image in overlay shows that the signal corresponding to FLP80 colocalizes only in the flagellar pocket near the kinetoplast with that of tomato lectin; the early endocytic compartment was labeled only by lectin.

### Example 3

### Vaccination Tests with the Flagellar Protein FLP80

Two groups of cattle were injected subcutaneously with the antigenic protein FLP80 mixed with two types of adjuvants, 1 mg/ml Quil A (saponin) and AdjuPhos (colloidal aluminum phosphate) volume to volume according to a final volume of 1 ml or just with the adjuvant mixture (control). One injection was given each three weeks for a total of three injections of 100 μg, 50 μg and 25 μg of antigen, respectively. The animals were infected by T. congolense strain IL3000 three weeks after the last injection in a ratio of 1,000 parasites per animal intradermally. Blood samples were taken daily until all the animals were recognized as infected, parasitemia being determined by buffy-coat analysis. Thereafter, weekly blood samples were taken to monitor parasitemia and anemia, and the animals were weighed monthly. The kinetics of the response to immunization and to infection were monitored by ELISA on the various immunizing antigens.

### Example 4

### Example of Diagnostic Tests on Infected Animal Blood

This test is carried out by detecting circulating antigen FLP80 by the sandwich ELISA method. The anti-FLP80 “capture” antibody is adsorbed in the wells of a 96-well plate by incubation overnight at 4° C. of 1-10 μg/ml of capture antibody diluted in 100 μl of 50 mM NaHCO₃ buffer (pH 9.6). The plate is then emptied and washed three times with 200 μl per well of PBS-Tween solution (3.2 mM Na₂HPO₄, 0.5 mM KH₂PO₄, 1.3 mM KCl, 135 mM NaCl (pH 7.4), 0.05% Tween 20). Next, 100 μl of blocking solution (0.2% gelatin in PBS-Tween) is added to each well and incubated for 30 minutes at room temperature. The plates are emptied and then 100 μl of animal sera to be tested is deposited in the wells and incubated for 2 hours at 37° C. The plate is then emptied and then washed three times with 200 μl per well of PBS-Tween solution. 100 μl of a solution containing the second antibody coupled to biotin (PBS-Tween containing 1-10 μg/ml of biotinylated antibody) is added to each well and incubated for 1 hour at 37° C. The plate is then emptied and then washed four times with 200 μl per well of PBS-Tween solution. 100 μl of PBS-Tween containing streptavidin coupled to peroxidase (Sigma) is added according to the manufacturer's recommendations. The plate is then emptied and then washed four times with 200 μl per well of PBS-Tween solution. Finally, the reaction is visualized by adding peroxidase substrate according to the manufacturer's recommendations (example of a developer substrate that can be used: ABTS (Sigma)). The result is read using a plate reader or fluorometer according to the manufacturer's recommendations.

The capture antibody used can be either an immunopurified polyclonal serum against T. congolense FLP80 protein, or a monoclonal antibody recognizing an epitope present on said protein. The second antibody is a monoclonal antibody different than the capture antibody which recognizes a different FLP80 epitope.

What is claimed:

1-31. (canceled)

32. DNA or RNA molecule comprising at least one nucleotide sequence coding for a flagellar pocket protein of an African trypanosome, wherein said molecule comprises the nucleotide sequence as represented in the sequence SEQ ID NO: 1, a sequence complementary to the sequence SEQ ID NO: 1, a sequence comprising an identity of at least 85% over the entire length of the sequence SEQ ID NO: 1, a fragment of said sequences comprising between 30 and 100 nucleotides, or a nucleotide sequence able to hybridize with the sequence SEQ ID NO: 1 under stringent hybridization conditions.

33. protein encoded by the nucleotide sequence of claim 32.

34. A protein which comprises the amino acid sequence such as represented in the sequence SEQ ID NO: 2, and which is designated FLP80, or an antigenic peptide fragment of said protein.

35. An expression cassette functional in a cell of prokaryotic or eukaryotic origin, comprising a DNA molecule of claim 32 and enabling expression of said molecule in such a cell.

36. A recombinant vector comprising a nucleic acid of claim 32.

37. A recombinant host cell comprising a nucleic acid of claim 32.

38. A protein of either claim 33 or claim 34, or an antigenic peptide fragment of said protein, characterized in that said protein or said fragment exhibits reactivity with the sera of nonhuman animals or humans infected by an African trypanosome.

39. A vaccine comprising an effective amount of one or more proteins of claim 33 or 34.

40. A method for preventing and/or treating trypanosomosis or pathogeneses induced by trypanosomosis in a non human animal, or for preventing and/or treating trypanosomiasis or pathogeneses induced by said trypanosomiasis in humans, comprising administering to said human or non-human subject a vaccine of claim 39.

41. A vaccine of claim 39, for protecting against infections by an African trypanosome selected from Trypanosoma congolense, Trypanosoma vivax, Trypanosoma evansi and/or Trypanosoma brucei, and preferably against infections by Trypanosoma congolense.

42. A method of claim 40, wherein said induced pathogeneses comprise anemia, degradations in general health, weight loss and/or immunosuppression in human or in nonhuman animals.

43. A method of claim 40, wherein said nonhuman animal is selected among bovids, ovids, felids, suids, camelids and/or canids.

44. A vaccine of claim 39, which is a multivalent vaccine further comprising one or more antigenic peptides and/or antigenic fragments and/or nucleotide sequences coding for said peptides derived from one or more African trypanosome species, preferably derived from flagellar proteins, sialidases, trans-sialidases, proteases, lipases and/or tubulins.

45. A vaccine of claim 39, which further comprises (i) at least one antiparasitic agent, preferably selected from a trypanocide and/or a nonspecific antiparasitic agent for trypanosomes, (ii) at least one anti-infective agent, preferably selected from β-lactams, fosfomycin, glycopeptides or polypeptides with antibiotic activity, bacitracin, aminoglycosides, macrolides, lincosamides, streptogramins, tetracyclines, phenicols, fusidic acid or quinolones, and/or (iii) at least one symptomatic agent, preferably selected from an anti-anemia agent, a hepatoprotective agent and/or a non-steroidal anti-inflammatory drug.

46. A vaccine of claim 39, characterized in that it further comprises an adjuvant.

47. A vaccine comprising the vaccine of claim 39 and a vaccine and/or or an antigen directed against theileriosis, anaplasmosis, babesiosis, foot-and-mouth disease, clostridiosis, plague, catarrhal fever, contagious bovine pleuropneumonia (CBPP), blackleg, pasteurellosis and/or sheep pox.

48. A monoclonal or polyclonal antibody which binds a protein of claim 33 or an antigenic peptide fragment of said protein, and/or which is obtained by immunological reaction of a nonhuman animal organism with at least one protein of claim 33 or an antigenic peptide fragment thereof.

49. A method for the in vitro diagnosis of trypanosomosis in a biological sample of a nonhuman animal able to be infected by an African trypanosome, wherein said sample and an antibody of claim 48 are brought together under conditions enabling a possible immunological reaction, and in that the presence or absence of an immune complex is then detected.

50. A kit for diagnosing trypanosomosis in a biological sample, comprising at least one antibody of claim 48, a medium suitable to the formation of an immune complex with said antibody and at least one reagent for detecting an immunological reaction.

51. A method for screening trypanocidal agents or trypanosome inhibitors, comprising bringing together a previously-radiolabeled agent to be tested with a cell expressing the protein of claim 33 or 34, and measuring the radioactivity associated with said cell.

52. A competitive inhibitor of FLP80 protein of claim 34, obtainable by the method of claim 51, for use in the treatment and/or the prevention of trypanosomosis, wherein said inhibitor is comprised of a truncated apolipoprotein A or E1.
